(19) United States
(12) Patent Application Publication
(10) Pub. No.: US 2010/0168255 A1
(43) Pub. Date: Jul. 1, 2010

(54) METHOD FOR PRODUCING A COMPOUND WHICH HAS AT LEAST ONE ETHER GROUP

(76) Inventors: Alfred Westfechtel, Hilden (DE); Elke Grundt, Dusseldorf (DE); Peter Daute, Beverstedt (DE); Norbert Klein, Mettmann (DE)

Correspondence Address: SMITH MOORE LEATHERWOOD LLP, P.O. BOX 21927, GREENSBORO, NC, 27420, US

(21) Appl. No.: 12/602,945

(22) PCT Filed: Jun. 11, 2008
(86) PCT No.: PCT/EP2008/004667

§ 371 Date: Mar. 2, 2010

(30) Foreign Application Priority Data
Jun. 11, 2007 (DE) 10 2007 027 371.3

(51) Int. Cl. A61K 8/37; C11C 3/00; C08K 5/10; A61Q 90/00; C09K 8/36

(52) U.S. Cl. 514/785; 554/168; 524/318; 507/138

(57) ABSTRACT

The present invention relates to a process for preparing a compound having at least one ether group, at least one ester group, at least one amino group or at least one urethane group by a1) preparing 1,2-propanediol by means of a process wherein glycerol is hydrogenated in the presence of a catalyst, glycerol being reacted to at most 95% and a 1,2-propanediol phase obtained and a2) reacting the 1,2-propanediol phase with a compound having at least one active hydrogen atom, at least one epoxide group, at least one ester group or at least one isocyanate group. The present invention relates to the compounds which can be obtained using this process and the chemical products, emulsions, cosmetic formulations, plastics material compositions and drilling compositions prepared using the compound and the use and preparations thereof.

The invention relates generally to a process for preparing a compound having at least one ether group, at least one ester group, at least one amino group or at least one urethane group or at least two thereof. The present invention also relates to the compound which can be obtained using this process and contains at least one ether group, at least one ester group or at least one urethane group, to the use of this compound in chemical products, to chemical products, to a process for preparing an emulsion and the emulsion obtainable using this process, to a cosmetic composition and a process for preparing this cosmetic composition, to plastic material compositions and the preparation thereof, to drilling compositions and the preparation thereof and to the use of a 1,2-propanediol phase.

Esters of organic acids, such as for example fatty acid esters, are frequently used as emulsifiers, in particular as emulsifiers in cosmetic compositions. In this case, the emulsifiers serve to improve the stability of emulsions. Particularly frequently, use is in this case made also of esters obtained by esterification of organic acids with 1,2-propanediol. In this case, the purity of the 1,2-propanediol used plays an important part, 1,2-propanediol having a purity of at least 98% by weight conventionally being used.

1,2-Propanediol is frequently prepared by the catalytic hydrogenation of glycerol or else by the catalytic hydration of propylene oxide; although the preparation of 1,2-propanediol by hydrogenation of glycerol has been described several times, to date it has not been able to become established in industrial practice.

DE-C-524 101 discloses the hydrogenation of glycerol, wherein according to Example 1 of this document a stream of hydrogen containing 1 to 1.5% by volume of glycerol is passed at 200 to 210° C. over a catalyst which is attached to glass beads. Obtained in this case is a liquid which is subjected to fractional distillation after removal of the water formed during the hydrogenation.

DE-C-541 362 describes the hydrogenation of polyoxy compounds. In this case, liquid or solid polyoxy compounds are treated in aqueous solution or suspension with hydrogen in the presence of catalysts. In Example 1 glycerol and a nickel catalyst are treated with hydrogen in a bomb at 200 to 240° C. and a pressure of 100 atm. The process described in DE-C-541 362 is a discontinuous batch process in which the glycerol used is reacted almost completely.

According to U.S. Pat. No. 5,616,817, the glycerol hydrogenation is carried out in such a way that glycerol having a water content of up to 20% by weight is used and specific catalysts (having a content of 40-70% Co, 10-20% Mn, 0-10% Mo and 0-10% Cu) are used for hydrogenation. According to the teaching of U.S. Pat. No. 5,616,817, the glycerol used is reacted completely.

As is apparent from the documents described hereinbefore, almost complete reaction of the glycerol is in all cases striven for in the preparation of 1,2-propanediol from glycerol. As, moreover, numerous by-products are formed over the course of the dehydrogenation, it is in addition conventional to purify, for example by means of distillation, the 1,2-propanediol phase obtained during the dehydrogenation in order thus to obtain 1,2-propanediol having a purity of at least 98% by weight.

If an ester, for example a fatty acid ester, is prepared using a 1,2-propanediol obtained in this way, the properties of such an ester are frequently disadvantageous. Especially when used as an emulsifier, esters of this type frequently have an unsatisfactory stabilizing effect.

The present invention was based on the object of at least partly overcoming the drawbacks resulting from the prior art.

In addition, the present invention was based on the object of specifying a process which can be used to prepare, in as few process steps as possible, ethers or esters, the alcohol components of which can be prepared from renewable raw materials or from educts which can be obtained from renewable raw materials. In addition, the ethers or esters which can be obtained using this process should have advantageous, but at least equally good, properties relative to conventional ethers or esters. In particular, when used as emulsifiers, they should allow better stability compared to conventional emulsifiers.

The present invention was also based on the object of providing ethers or esters which allow, when used as an emulsifier, noticeably better stabilization of an emulsion compared to the ether or ester compounds known in the art.

Furthermore, the present invention was based on the object of providing an emulsion which can serve for example as the basis for a cosmetic composition, this emulsion being characterized by better stability compared to the emulsions known in the art. The emulsions should also be characterized by a better odorous quality compared to the emulsions which are known in the art and contain an ester of 1,2-propanediol as the emulsifier.

The present invention was also based on the object of providing ether or ester compounds which are based on 1,2-propanediol and also have no unpleasant and/or strong odor, but rather have at most a mild odor and preferably should be odorless.

A contribution to achieving at least one of the objects mentioned hereinbefore is provided by the subject-matters of the statutory-class forming claims, the sub-claims which are dependent thereon representing further embodiments according to the invention.

A contribution to achieving the objects mentioned hereinbefore is provided by a process for preparing a compound having at least one ether group, at least one ester group, at least one amino group, at least one urethane group or at least two thereof, preferably having at least one ester group, including the process steps
a1) preparing 1,2-propanediol by means of a process wherein glycerol is preferably continuously hydrogenated, preferably in the presence of a heterogeneous, particularly preferably a heterogeneous, copper-containing catalyst, most preferably a heterogeneous, copper-chromium-containing catalyst, glycerol being reacted to at most 95% by weight, preferably at most 90% by weight, even more preferably at most 85% by weight, additionally preferably at most 80% by weight, additionally even more preferably to at most 75% by weight and most preferably to at most 70% by weight and a 1,2-propanediol phase is obtained;
a2) reacting the 1,2-propanediol phase obtained in process step a1) with a compound having at least one active hydrogen atom, at least one epoxide group, at least one ester group or at least one isocyanate group.

Furthermore, in accordance with a preferred embodiment, in process step a1) glycerol is reacted to at least 30% by weight, preferably to at least 40% by weight, or to at least 50% by weight, further preferably to at least 60% by weight, or to at least 65% by weight, and even more preferably to at least 70% by weight, or most preferably to at least 80% by weight, and a 1,2-propanediol phase obtained. Particularly preferably, glycerol is reacted in process step a1) in a range of from 50 to 95% by weight, or from 60 to 95% by weight, or from 70 to 90% by weight.

The percentages by weight used in the present document result from gas chromatography analyses by integrating the surface areas of the signals and relate to the total weight of the composition measured as a sample. For calibrating and checking the measurements, the percentages by weight were confirmed by distillation and weighing of the individual components from a sample measured by gas chromatography.

In a further configuration of the process according to the invention, low boiler or water separating-off or separating-off from water and low boilers can take place. This separating-off or this combination of separatings-off can take place as variants in step a1), after step a1), prior to step a2), in step a2) or after step a2) or in a combination of at least two of these variants. Examples of low boilers include in particular substances which boil below the boiling temperature of 1,2-propanediol.

A compound having at least one active hydrogen atom in this case preferably means a compound having at least one hydrogen atom which is bound to an atom different from carbon, preferably to an oxygen atom, a nitrogen atom or a sulphur atom, particularly preferably to an oxygen atom or a nitrogen atom and most preferably to an oxygen atom. These compounds having at least one active oxygen atom therefore have preferably an OH group, a COOH group, an NH₂ group, an NRH group (wherein R is a further organic residue, such as for example an alkyl or alkenyl group) or an SH group.

It has been found surprisingly, but none the less advantageously, that compounds such as for example esters, polyethers, ethers, polyethers, ester polyethers or else polyurethanes having 1,2-propanediol as an alcohol component can be obtained with advantageous properties if use is made, for preparing these compounds, not of pure 1,2-propanediol, but rather of a 1,2-propanediol phase which was obtained by merely partial hydrogenation of glycerol. Especially the esters obtained as a result of use of this 1,2-propanediol phase are capable, compared to those products which were obtained as a result of the use of particularly pure 1,2-propanediol, of stabilizing emulsions for much longer.

In process step a1) of the process according to the invention, glycerol is continuously hydrogenated in the presence of a heterogeneous, preferably a copper-containing catalyst, particularly preferably a heterogeneous, copper and chromium-containing catalyst, glycerol being reacted to at most 95%, preferably at most 90%, even more preferably at most 85%, additionally preferably at most 80%, additionally even more preferably to at most 75% and most preferably to at most 70% and a 1,2-propanediol phase being obtained.

It is in this case particularly preferred that the hydrogenation is carried out in the presence of less than 20% by weight, particularly preferably less than 10% by weight, even more preferably less than 5% by weight, based on the amount by weight of glycerol used, of an organic solvent, carrying-out of the process in the complete absence of an organic solvent being most preferred. Often, the hydrogenation is carried out in the presence of at least 1, preferably at least 2% by weight of an organic solvent.

Examples of catalysts include in process step a1) full and carrier contacts containing as their main component metals, metal salts or metal oxides or the like from subgroups I and VIII. Further metals can be added as dopants to improve the properties.

The catalyst can in this case have been produced in different ways including precipitation of the metal salts, impregnation, ion exchange or solid-state reactions, to name but a few examples.

The catalyst used can be in the form of the hydrogenation catalysts which are known per se and are used for example in the preparation of fatty alcohols from fatty acid methyl esters or in the hardening of fatty acid. In particular, it is however proposed to carry out the process using catalysts having copper as their active component, Cu chromite, Cu zinc oxide, Cu aluminum oxide or Cu silicon dioxide being particularly preferred and Cu chromite catalysts being most preferred.

The Cu chromite catalyst which is preferably used in this connection contains 35 to 55% by weight, preferably 40 to 50% by weight of copper, 35 to 55% by weight, preferably 40 to 50% by weight of chromium, based in each case on the oxidic catalyst mass, and optionally further alkaline-earth or transition metals, in particular barium and manganese, in the form of the oxides thereof. It is in this case beneficial if the catalyst contains 1 to 7% by weight, in particular 1.5 to 3% by weight of barium, based on the oxidic catalyst mass. As an Example of a suitable catalyst a catalyst shall be mentioned containing approximately 47% by weight of CuO, 46% by weight of Cr₂O₃, 4% by weight of MnO₂ and 2% by weight of BaO. This catalyst and the process for the production thereof are described in detail in EP 254 189 A2. Reference is hereby expressly made to the disclosure of the catalyst and the process for the production thereof contained in EP 254 189 A2, and is incorporated herein by reference. The invention is not however limited to Cu chromite catalysts. Other catalysts, such as for example Cu/ZnO catalysts or Cu/Al₂O₃ catalysts, can also be used. Catalysts which are suitable for the process according to the invention are commercially available from Südchemie AG, Germany, and Engelhard Inc., USA.

It is furthermore preferred that the catalyst has a high surface area and porosity, thus providing high activity and selectivity and a long service life which is particularly important for technical applications. Thus, it is advantageous if the catalyst used has a specific surface area in the range of from 20 to 100 m²/g, preferably 70 to 80 m²/g.

Process step a1) of the process according to the invention can be carried out continuously and discontinuously, the continuous procedure being particularly preferred. In the case of a discontinuous procedure, a predetermined amount of glycerol is placed into a reactor and is then dehydrogenated by being brought into contact with hydrogen in the presence of the catalyst under the required reaction conditions (pressure, temperature, etc.). A process of this type is for example described in DE-C-541 362. In the case of the continuous procedure, a gas phase containing hydrogen and glycerol, which can be present in vaporous or liquid form, is passed preferably continuously over a catalyst fixed bed. In this case, the procedure is preferably such that diluted or undiluted hydrogen is used for hydrogenation, operation being carried out preferably at pressures in the range of from 20 to 300 bar, in particular at 100 to 250 bar, and at temperatures in the range of from 150° C. to 280° C., in particular 180 to 220° C. Preferably, a molar ratio of hydrogen to glycerol is set in the range from 2 to 500, particularly preferably 10 to 350, excess hydrogen optionally being circulated. This means that the amount of hydrogen gas throughput, measured in mol of H₂/hour, is 2 to 500 times higher than the amount of glycerol throughput, measured in mol of glycerol/hour. A most preferred range of the reaction ratio is 30:1 to 200:1.

Process step a1) of the process according to the invention can be carried out in reactors which are similar to those known reactors which are conventional for the preparation of fatty alcohols by hydrogenation of fatty acid methyl ester or directly from triglycerides and which are preferably fixed-bed reactors. Preferably, process step a1) of the process according to the invention is carried out in tubular reactors or multitube fixed-bed reactors operated under isothermal conditions. Also conceivable is the use of reactors having heat exchange plates as constructional elements. Both in the tubular reactors and in the reactors having heat exchange plates, the catalyst can be introduced in the form of a catalyst fixed-bed charge or else be attached as a coating to the inside of the tubes or heat exchange plates. The reaction parameters, temperature and pressure, can in this case be adapted accordingly to the respective catalyst activity. Most of the reaction heat is dissipated via the reactor wall (in the case of a use of multitube fixed-bed reactors via the walls of the reaction tubes used, and in the case of a use of reactors with heat exchange plates via the heat exchange plates), so that practically isothermal operation is possible. If cooling of the reactor is not possible via air, then the reactor can optionally be cooled using a suitable coolant, this coolant flowing, when use is made of multitube fixed-bed reactors, along the reaction tubes and, when use is made of reactors with heat exchange plates, through the flow paths within the heat exchange plates. Suitable coolants include for example water, heat transfer liquids such as Marlotherm® or molten salts. Furthermore, it is particularly preferred that the hydrogenation is carried out in such a way that liquid glycerol is passed in trickle bed operation in parallel or countercurrent flow with hydrogen over a catalyst fixed bed. In a further embodiment using reaction tubes, glycerol is passed through the catalyst charge in the reaction tube or tubes under measures at least partly preventing backmixing at an LHSV (“Liquid Hourly Space Velocity”), expressed in m³/h of glycerol per m³ of catalyst volume, in a range of from 0.1 to 20 h⁻¹, preferably in a range of from 0.1 to 5 h⁻¹, even more preferably in a range of from 0.2 to 3 h⁻¹ and additionally preferably in a range of from 0.3 to 2 h⁻¹. Examples of measures at least partly preventing backmixing include in principle all measures which are known to a person skilled in the art and seem suitable to him for this purpose, such as suitable tube cross sections or tube cross section/length ratios which are usually selected as a function of the flow conditions conventionally prevailing during operation of the reactor.

Further details concerning an advantageous configuration of the catalyst used for hydrogenation may be inferred inter alia from EP-A-0 334 118, the disclosure of which, in particular with regard to the avoidance of backmixing in the reactor, the advantageous volume loading of the reactor and the advantageous surface loading of the reactor, is incorporated herein by reference.

In addition to the use of a single fixed-bed reactor as a hydrogenation reactor, at least two interconnected fixed-bed reactors can also be used, the hydrogen being in this case passed through the at least two fixed-bed reactors in succession, without the gas which issues from the reactors and enters the subsequent reactors being cooled. A process of this type is for example described in EP-A-0 515 485, the disclosure of which with regard to the precise carrying-out of the hydrogenation process when use is made of two interconnected fixed-bed reactors is incorporated herein by reference.

In a further configuration of the present invention, the hydrogenation can be carried out in at least one reaction unit containing a reactor which is not operated under isothermal conditions and is connected to a cooler. The hydrogenation can take place in at least one or else 2 or more, often two to ten successive reaction units. These reaction units have at least one reactor which is cooled little or not at all and therefore operates non-isothermally, is often configured as a tube or bundle of tubes and is followed by a cooling region. The cooling region can be of a form which is generally known and seems suitable to a person skilled in the art such as a tubular or plate-type heat exchanger and also be cooled with any coolant which is familiar and seems suitable to a person skilled in the art. Examples of coolants thus include those mentioned hereinbefore. In one specific configuration, the cooling is carried out by a cooling gas which can contain hydrogen and such as is described in DE 198 43 798 A1.

The glycerol used for hydrogenation can contain water. Preferably, the water content should however be below 15% by weight, particularly preferably below 10% by weight, additionally preferably below 5% by weight and most preferably below 2% by weight, based in each case on the total weight of water and glycerol. It may also be desirable to use anhydrous glycerol or a glycerol containing only small traces of water.

In process step a2) of the process according to the invention, the 1,2-propanediol phase obtained in process step a1) is reacted with a compound having at least one active hydrogen atom, at least one epoxide group, at least one ester group or at least one urethane group.

The compound having at least one active hydrogen atom can be a compound having at least one hydroxyl group, so that after reaction of this compound with the 1,2-propanediol phase a compound having at least one ether group is obtained. Depending on the molar ratio at which the compound having at least one hydroxyl group is reacted with 1,2-propanediol contained in the 1,2-propanediol phase, a polyether compound can also be obtained.

The compound having at least one active hydrogen atom can also be a compound having at least one carboxylic acid group, so that after reaction of this compound with the 1,2-propanediol phase a compound having at least one ester group is obtained. Depending on the molar ratio at which the compound having at least one carboxyl group is reacted with the 1,2-propanediol contained in the 1,2-propanediol phase, an ester polyether compound can also be obtained.

The compound having at least one active hydrogen atom can also be a compound having at least one amino group, so that after reaction of this compound with the 1,2-propanediol phase a compound having at least one amino group and at least one hydroxyl group is obtained. Depending on the molar ratio at which the compound having at least one amino group is reacted with 1,2-propanediol contained in the 1,2-propanediol phase, an aminopolyether compound can also be obtained.

If a compound having at least one epoxide group is used in process step a2), then ether or polyether can also be obtained by reacting this compound with the 1,2-propanediol phase.

If a compound having at least one isocyanate group is used in process step a2), then urethanes or polyurethanes can be obtained by reacting this compound with the 1,2-propanediol phase.

Particularly preferred according to the invention is the use of a compound having at least one carboxylic acid group in process step a2). In this case, the compound having at least one carboxylic acid group can be a mono carboxylic acid, a dicarboxylic acid or a tricarboxylic acid, monocarboxylic and dicarboxylic acids being particularly preferred and monocarboxylic acids, in particular fatty acids, most preferred. Furthermore, it is preferred that the compound having at least one carboxylic acid has 5 to 30, particularly preferably 10 to 25 and most preferably 15 to 20 carbon atoms per molecule. In this connection, it is particularly preferred that the compound having at least one carboxylic acid group is a C₅ to C₃₀ monocarboxylic acid, additionally preferably a C₁₋₁₀ to C₂₅ monocarboxylic acid and most preferably a C₁₅ to C₂₀ monocarboxylic acid, the above-mentioned monocarboxylic acids being saturated monocarboxylic acids, such as for example caproic acid, heptanoic acid, caprylic acid, pelargonic acid, capric acid, lauric acid, myristic acid, fish oil, palmitic acid, margaric acid, stearic acid, arachic acid, behenic acid, lignoceric acid or cerotic acid, singly unsaturated monocarboxylic acids such as for example undecylenic acid, oleic acid, elaidic acid, vaccenic acid, eicosenoic acid, cetoleic acid, erucic acid or nervonic acid, or multiply unsaturated monocarboxylic acids, such as for example linoleic acid, linolenic acid, arachidonic acid, timnodonic acid, clupanodonic acid or cervonic acid.

The above-mentioned fatty acids can be obtained for example from vegetable oils, hydrogenated vegetable oils, marine oils and animal fats and oils. Preferred vegetable oils include corn oil, canola oil, olive oil, cotton seed oil, soya oil, sunflower oil, high-erucic acid rapeseed oil, partially or completely hydrogenated soya oil, partially or completely hydrogenated canola oil, partially or completely hydrogenated sunflower oil, rapeseed oil, in particular partially or completely hydrogenated high-erucic acid rapeseed oil and partially or completely hydrogenated cotton seed oil, palm oil or palm stearin.

Examples of a dicarboxylic acid include in particular compounds selected from the group consisting of phthalic anhydride; isophthalic acid; terephthalic acid; tetrahydrophthalic anhydride; hexahydrophthalic anhydride; naphthalinedicarboxylic acid; 4,4′-biphenyldicarboxylic acid; diphenylmethane-4,4′-dicarboxylic acid; succinic acid; fumaric acid; adipic acid; sebacic acid; azelaic acid and maleic anhydride, of which adipic acid, terephthalic acid or azelaic acid are preferred and terephthalic acid is particularly preferred.

The compound having at least one carboxylic acid group is reacted in process step a2) of the process according to the invention as the acid component with 1,2-propanediol as the alcohol component so as to obtain an ester. In this case, it is however in principle also possible to use 1,2-propanediol not as the single alcohol component, but rather additionally to use at least one further alcohol component, so that a compound having at least two different ester groups is obtained. This at least one further alcohol component can be a tetrahydric or polyhydric alcohol, such as for example diglycerol, triglycerol, polyglycerol, pentaerythritol, dipentaerythritol or sorbitol, or else a trihydric, dihydric or monohydric alcohol, such as for example trimethylolpropane, trimethylolethane, a dihydric alcohol such as for example ethylene glycol, diethylene glycol, triethylene glycol, 1,3-propanediol, 1,4-butanediol, 1,5-pentanediol, neopentyl glycol, 1,6-hexanediol, 1,4-dimethylolcyclohexane, methanol, ethanol, 1-propanol or 2-propanol. Insofar as at least one further alcohol component is used, it is however preferred that the proportion formed by this further alcohol component of the total amount of 1,2-propanediol and further alcohol component is at most 50% by weight, particularly preferably at most 25% by weight, additionally preferably at most 10% by weight and most preferably at most 5% by weight.

According to one particular embodiment of the process according to the invention, the 1,2-propanediol phase obtained in process step a1) is reacted with a fatty acid so as to obtain a fatty acid ester.

In this case, it is preferred that the compound containing at least one carboxylic acid group is reacted with the alcohol component in an amount such that the molar ratio of carboxylic acid groups:hydroxyl groups is in a range of from 1:1.2 to 1:5, particularly preferably in a range of from 1:1.5 to 1:2 and most preferably in a range of from 1:1.7 to 1:1.9.

Furthermore, it is preferred that the fatty acid is reacted with the alcohol component, i.e. with the 1,2-propanediol or a mixture of the 1,2-propanediol and at least one further alcohol in the presence of an esterification catalyst. The esterification catalysts used can be acids, such as for example sulphuric acid or p-toluenesulphonic acid, or metals and the compounds thereof. Suitable are for example tin, titanium, zirconium which are used as finely divided metals or expediently in the form of their salts, oxides or soluble organic compounds. The metal catalysts are, in contrast to protonic acids, high-temperature catalysts which generally achieve their full activity only at temperatures above 180° C. They are however preferred according to the invention, because they supply fewer by-products, such as for example olefins, than proton catalysis. Esterification catalysts which are particularly preferred in accordance with the invention are one or more divalent tin compounds or tin compounds or elemental tin which can be reacted with the educts to form divalent tin compounds. For example, the catalyst used can be tin, tin (II) chloride, tin (II) sulphate, tin (II) alcoholates or tin (II) salts of organic acids, in particular of monocarboxylic and dicarboxylic acids. Particularly preferred tin catalysts are tin (II) oxalate and tin (II) benzoate.

The esterification reaction can be carried out using the process known to a person skilled in the art. In this case, it can be particularly advantageous to remove the water formed during the reaction and the water which may originate from the 1,2-propanediol phase from the reaction mixture, this removal of the water being carried out preferably by distillation, optionally by distillation with excess 1,2-propanediol. 1,2-Propanediol which has not reacted after carrying out the esterification reaction can also be removed from the reaction mixture, this removal of the 1,2-propanediol also being carried out preferably by means of distillation. Furthermore, after completion of the esterification reaction, in particular after the separating-off of non-reacted 1,2-propanediol, the catalyst remaining in the reaction mixture can be separated off, optionally after treatment with a base, by filtration or by centrifugation.

Furthermore, it is preferred to carry out the esterification reaction at a temperature in a range of from 50 to 300° C., particularly preferably in a range of from 100 to 250° C. and most preferably in a range of from 150 to 200° C. The optimum temperatures depend on the alcohol(s) used, the progress of the reaction, the type of catalyst and the catalyst concentration. They can easily be determined for each individual case by carrying out tests. Elevated temperatures increase the reaction speeds and promote secondary reactions, such as for example the splitting-off of water from alcohols or the formation of colored by-products. The desired temperature or the desired temperature range can be set by way of the pressure in the reaction vessel (slight excess pressure, normal pressure or optionally reduced pressure).

According to another particular embodiment of the process according to the invention, the 1,2-propanediol phase obtained in process step a1) is reacted with a dicarboxylic or tricarboxylic acid and a fatty acid so as to obtain an alkyd resin.

Alkyd resins are synthetic, highly hydrophobic polymers obtained by condensation of diols (in the present case of 1,2-propanediol) with polybasic acids with the addition of organic oils or fatty acids (to modify the properties of the resin) and optionally further, polyhydric alcohols, in particular glycerol or pentaerythritol. In this case, it is particularly preferred that the compound having at least one carboxylic acid group is a dibasic acid which is preferably selected from the group consisting of phthalic anhydride, isophthalic acid, terephthalic acid, tetrahydrophthalic anhydride, hexahydrophthalic anhydride, naphthalinedicarboxylic acid, 4,4′-biphenyldicarboxylic acid, diphenylmethane-4,4′-dicarboxylic acid, succinic acid, fumaric acid, adipic acid, sebacic acid, azelaic acid and maleic anhydride, of these adipic acid, terephthalic acid or azelaic acid being preferred and terephthalic acid particularly preferred.

Examples of organic oils include in the first place oils or oil derivatives of vegetable and animal origin, individually or as a mixture in each case. Examples of oils or oil derivatives of animal origin include tallows and oleic acids obtained from tallows, in particular by fractionation. Examples of organic oleic or fatty acids include in particular tallows, canola oil, rape oil, sunflower oil, palm oil, which can optionally be present also in hardened or semi-hardened form, soya bean oil, thistle oil, linseed oil, tall oil, coconut oil, palm kernel oil, castor oil, dehydrogenated castor oil, fish oil and tung oil. Particularly preferred are drying oils or semidrying oils having iodine values of at least 100; inter alia soya bean oil and tall oil are advantageous. Examples of fatty acids used both for preparing the alkyd resins and for preparing the fatty acid esters include in particular those of soya bean oil, thistle oil, linseed oil, tall oil, coconut oil, palm kernel oil, castor oil, dehydrogenated castor oil, fish oil and tung oil. Of these fatty acids, those of drying oils or semidrying oils having iodine values of at least 100, inter alia those of soya bean oil and tall oil, are preferred.

The preparation of alkyd resins may be inferred for example from WO-A-01/62823, the disclosure of which with regard to the preparation of alkyd resins is incorporated herein by reference.

Also conceivable is the use of a compound having at least one hydroxy group in process step a2). In this case, the compound having at least one hydroxy group can be a monool, a diol, a triol or an alcohol having more than three OH groups. Particularly preferred compounds having at least one OH group are however fatty acid alcohols which were obtained by reduction of fatty acid esters, for example with sodium in a Bouveault-Blanc reaction or hydrogenation under pressure. Fatty alcohols which are suitable in this connection are for example hexanol, octanol, decanol, dodecanol, tetradecanol, hexadecanol, heptadecanol, octadecanol, eicosanol, behenyl alcohol, delta-9-cis-hexadecenol, delta-9-octadecenol, trans-delta-9-octadecenol, cis-delta-11-octadecenol or octadecatrienol.

The preparation of ethers from fatty alcohols and 1,2-propanediol, in particular the preparation of polyethers by the polyproxylation of fatty alcohols with 1,2-propanediol is carried out also preferably by means of suitable catalysts, such as calcium and strontium hydroxides, alkoxides or phenoxides (EP-A-0 092 256), calcium alkoxides (EP-A-0 091 146), barium hydroxide (EP-B-0 115 083), basic magnesium compounds, such as for example alkoxides (EP-A-0 082 569), magnesium and calcium fatty acid salts (EP-A-0 085 167), antimony pentachloride (DE-A-26 32 953), aluminum isopropylate/sulphuric acid (EP-A-0 228 121), zinc, aluminum and other metal-containing oxo compounds (EP-A-0 180 266) or aluminum alcohols/phosphoric acids (U.S. Pat. No. 4,721,817). More precise information for preparing polypropxylated fatty alcohols can be inferred inter alia also from EP-A-0 361 083, the disclosure of which with regard to the preparation of polyethers from 1,2-propanediol and fatty alcohols is incorporated herein by reference.

Also conceivable is the use of a compound having at least one amino group in process step a2). In this case, the compound having at least one amino group can in particular be a fatty amine which can be obtained for example from triglycerides by treatment with ammonia and subsequent hydrogenation. The propoxylation of amines with 1,2-propanediol is also described in EP-A-0 361 083, to which reference is hereby made.

When use is made of a compound having at least one epoxide group in process step a2), the 1,2-propanediol phase is reacted with compounds such as for example ethylene oxide, propylene oxide, ethylene diglycidyl ether, propylene diglycidyl ether, diethylene diglycidyl ether, dipropylene diglycidyl ether, triethylene diglycidyl ether, tripropylene diglycidyl ether, tetraethylene diglycidyl ether, tetrapropylene diglycidyl ether or polyethylene diglycidyl ethers or polypropylene diglycidyl ethers having a still higher molecular weight, wherein polyethers can also be obtained.

When use is made of a compound having at least one ester group in process step a2), the reaction with 1,2-propanediol results in transesterification, esters of the above-mentioned mono fatty acids preferably being used as the ester having at least one ester group.

When use is made of a compound having at least one isocyanate group in process step a2), the 1,2-propanediol phase is reacted preferably with diisocyanates, such as for example hexamethylene diisocyanate (HDI), isophorone diisocyanate (IPDI), toluoylene diisocyanate (TDI), diphenylmethane diisocyanate (MDI) or mixtures of diphenylmethane diisocyanate and polymethylene polyphenylene polyisocyanates, in the presence of suitable catalysts. A suitable process for preparing polyurethanes is for example that in DE-A-10 2004 041 299, the disclosure of which with regard to the preparation of polyurethanes from diols and polyisocyanates is incorporated herein by reference.
According to a further preferred embodiment, the a1-1,2-propanediol phase obtained in process step a1) contains glycerol contamination in a range of from 0.01 to 20% by weight, or from 0.1 to 15% by weight, or from 1 to 10% by weight, based in each case on the total amount of the a1-1,2-propanediol phase,
   and
   the a2-1,2-propanediol phase used in process step a2) contains at least 70 up to 100% by weight, or at least 75 up to 100% by weight, or at least 80 up to 100% by weight, or at least 70 to 95% by weight, of the glycerol contamination of the a1-1,2-propanediol phase.

In accordance with the foregoing, the a1-1,2-propanediol phase obtained in process step a1) can be used in process step a2) as the a2-1,2-propanediol phase substantially without intermediate treatment or purification steps.

In this case, it is generally not necessary to subject the a1-1,2-propanediol-containing phase to thermal separation processes in order to separate off all of the impurities contained. Nevertheless, it is possible, for example by applying a suitable vacuum, to remove a portion of the glycerol contamination, or else portions of further contaminations such as for example monoalcohols. Occasionally, the a1-1,2-propanediol phase obtained in process step a 1) can be passed, for use as the a2-1,2-propanediol phase, over a gently heated wiring section placed under reduced pressure. Equally, it may prove advantageous to separate off at least solids, such as for example catalysts, this being carried out preferably using non-thermal separation processes such as filtration, sedimentation or centrifugation.

According to a further preferred embodiment, the ratio of glycerol to 1,2-propanediol in the 1,2-propanediol phase obtained in process step a1) is in a range of from 1:3 to 1:8.

According to a particularly preferred embodiment of the process according to the invention, the 1,2-propanediol phase obtained in process step a1) is not purified, preferably is at least not purified by thermal separation processes, such as for example distillation or rectification, prior to the reaction in process step a2). The 1,2-propanediol phase is therefore supplied directly to the reaction with a compound having at least one active hydrogen atom, at least one epoxide group, at least one ester group or at least one isocyanate group. Nevertheless, it can prove advantageous to separate off from the 1,2-propanediol-containing phase at least solids, such as for example catalysts, this separating-off being carried out preferably by way of non-thermal separation processes, i.e. for example by filtration, sedimentation or centrifugation.

A contribution to achieving the objects mentioned at the outset is provided also by a compound having at least one ether group, at least one ester group or at least one urethane group, preferably having at least one ester group which is obtainable, preferably was obtained, by way of the process according to the invention described hereinbefore. In this case, it is particularly preferred that this compound is a mono fatty acid ester which was obtained by reacting the 1,2-propanediol phase obtained in process step a1) with a fatty acid, preferably with a mono fatty acid.

A contribution to achieving the objects mentioned at the outset is provided also by the use of the above-described compound having at least one ether group, at least one ester group or at least one urethane group, preferably having at least one ester group, in chemical products. Examples of chemical products include in this case, in particular, cosmetic compositions or paints. In particular, the use of the compound according to the invention containing at least one ester group as an emulsifier or as a formulation component for PVC extrusions helps to achieve the objects mentioned at the outset.

A contribution to achieving the objects mentioned at the outset is provided in particular also by chemical products containing the above-described compound having at least one ether group, at least one ester group or at least one urethane group, preferably having at least one ester group.

These chemical products are characterized, compared to the products which are known in the art and also contain chemical components which were obtained by the use of 1,2-propanediol as an educt, by much lower odor pollution compared to the products obtained from complete or excessive hydrogenation. If the chemical product is an emulsion containing a mono fatty acid ester according to the invention as the emulsifier, then these emulsions are characterized by much better stability in storage compared to conventional emulsions.

A contribution to achieving the objects mentioned at the outset is provided in particular also by a process for preparing an emulsion, wherein an aqueous phase and an organic phase are brought into contact with each other in the presence of a compound according to the invention having at least one ester group, preferably in the presence of a mono fatty acid ester according to the invention so as to form an emulsion.

Examples of an organic phase include in this case in principle all organic solvents which are non-miscible with water. Particularly preferred organic phases are however based on synthetic, vegetable or animal oils. Examples of oils include in this case fatty acid triglycerides which are in a liquid state at room temperature, in particular vegetable oils such as for example rapeseed oil, sunflower oil, thistle oil, olive oil, linseed oil, pumpkin seed oil, hemp oil or mustard oil, or animal oils such as for example tallows, fish oil or neatsfoot oil, which are used relatively rarely as foods. They are however used in the chemical industry (for example lubricating oils, soaps) or in mechanical engineering (lubricants). Examples of synthetic oils include silicone oils. Hydrocarbon-based organic phases can also be contained in the emulsions. Examples of these include in particular paraffins.

Furthermore, the emulsion according to the invention can be a water-in-oil or an oil-in-water emulsion.

The amount of the compound according to the invention having at least one ester group, preferably the amount of a mono fatty acid ester according to the invention, in the emulsion according to the invention is preferably in a range of from 50 to 0.1% by weight, particularly preferably in a range of from 20 to 0.5% by weight and most preferably in a range of from 5 to 1% by weight, based in each case on the total weight of the emulsion. The ratio by volume of the aqueous phase and organic phase can fluctuate widely and is in particular also dependent on whether a water-in-oil or an oil-in-water emulsion is to be obtained.

The emulsion according to the invention is prepared preferably using the processes known to a person skilled in the art for preparing emulsions. Preferably, the individual components of the emulsion according to the invention are combined and emulsified by means of a suitable homogenization device, for example by means of a rapid stirrer, a high-pressure homogenizer, a shaker, a vibration mixer, an ultrasonic generator, an emulsifying centrifuge, a colloid mill or an atomizer.

A contribution to achieving the objects mentioned at the outset is provided furthermore by an emulsion which is obtainable using the process described hereinbefore. Compared to the emulsions known in the art, this emulsion is characterized by noticeably better stability in storage.

In another configuration, the invention relates to a cosmetic composition containing an emulsion according to the invention. A cosmetic composition according to the invention is obtained by a process, wherein an emulsion is prepared using a process according to the invention and brought into contact with at least one cosmetic component. Examples of cosmetic components include all ingredients known to a person skilled in the art for various cosmetic products such as ointments, pastes, creams, lotions, powders, waxes or gels and shampoos, soaps, facial and body scrubs, sunscreen agents or make-ups. Often, one or more of these emulsions are used in an amount in a range of from 0.01 to 15% by weight, preferably in a range of from 0.1 to 15% by weight and particularly preferably in a range of from 0.15 to 5% by weight, based in each case on the cosmetic composition.

In a further configuration, the invention relates to a plastics material composition containing at least one plastics material and a compound, containing at least one ether group according to the invention, at least one ester group according to the invention, at least one amino group according to the invention or at least one urethane group according to the invention, preferably containing at least one ester group according to the invention. Often, one or more of these compounds are used in amounts in a range of from 0.01 to 15% by weight, preferably in a range of from 0.1 to 15% by weight and particularly preferably in a range of from 0.15 to 5% by weight, based in each case on the plastics material. A contribution to the present invention is provided also by a process for preparing a plastics material composition, wherein a compound containing at least one ether group according to the invention, at least one ester group according to the invention, at least one amino group according to the invention or at least one urethane group according to the invention, preferably containing at least one ester group according to the invention, which are prepared in each case using a process according to the invention, is brought into contact with at least one plastics material. Examples of plastics materials include in principle all materials familiar to a person skilled in the art, preference being given to thermoplastic polymers, the processability of which, in particular the demoldability and wall adhesion of which, can be improved (Gächter/Müller, Kunststoff-Additive, Carl Hanser Verlag 1989). Preferred examples of these are polyesters and polyolefins. Preferred polyesters include PET, PBT, PLA or PHB. Preferred polyolefins include PE, PP, PVC, SAN, PVC being particularly preferred.

In another configuration, the invention relates to a drilling composition containing at least one liquid flushing component and a compound containing at least one ether group obtainable in accordance with the invention, at least one ester group obtainable in accordance with the invention, at least one amino group obtainable in accordance with the invention or at least one urethane group obtainable in accordance with the invention, preferably containing at least one ester group obtainable in accordance with the invention. Often, one or more of these compounds are used in an amount in a range of from 0.01 to 15% by weight, preferably in a range of from 0.1 to 15% by weight and particularly preferably in a range of from 0.15 to 5% by weight, based in each case on the flushing component.

A further configuration of the present invention relates to a process for preparing a drilling composition, wherein a compound containing at least one ether group, at least one ester group, at least one amino group or at least one urethane group, preferably containing at least one ester group, or else at least two thereof is prepared using a process according to the invention and is brought into contact with at least one liquid flushing component.

Examples of drilling compositions include in principle all compositions known to a person skilled in the art in particular for sinking bores in rock, in particular liquid flushing systems, drilling fluids on a continuous oil basis and drilling fluids based on water-based O/W emulsion systems.

It is known that liquid flushing systems for sinking bores in rock and bringing up the rock cuttings are slightly thickened, fluid systems which may be assigned to one of the following three classes: purely aqueous drilling fluids, oil-based drilling fluid systems which are generally used as what are known as invert emulsion muds, and water-based O/W emulsions containing a heterogeneous finely dispersed oil phase in the continuous aqueous phase.

Drilling fluids on a continuous oil basis are generally made up of a three-phase or multiphase system, namely: oil, water and fine-particle solids. The aqueous phase is in this case distributed in heterogeneous fine dispersion in the continuous oil phase. A plurality of additives are provided, in particular emulsifiers, fluid loss additives, alkali reserves, viscosity regulators and the like. With regard to the details, reference is made for example to the publication by P. A. Boyd et al. entitled “New Base Oil Used in Low-Toxicity Oil Muds” in Journal of Petroleum Technology, 1985, 137 to 142 and to R. B. Bennett “New Drilling Fluid Technology—Mineral Oil Mud” in Journal of Petroleum Technology, 1984, 975 to 981 and to the literature cited therein.

Drilling fluids based on water-based O/W emulsion systems occupy an intermediate position between the purely aqueous systems and the oil-based invert drilling fluids in terms of their use properties. Detailed information can be found in the relevant specialist literature, cf. for example the textbook by George R. Gray and H. C. H. Darley entitled “Composition and Properties of Oil Well Drilling Fluids”, 4^{th} Edition, 1980/81, Gulf Publishing Company, Houston and the extensive specialist and patent literature cited therein and the manual “Applied Drilling Engineering” by Adam T. Bourgoyne, Jr. et al., First Printing, Society of Petroleum Engineers, Richardson, Tex. (USA).

A further contribution to achieving the objects mentioned at the outset is provided by the use of the 1,2-propanediol phase obtained in process step a1) of the process according to the invention for preparing a compound having at least one ether group, at least one ester group, at least one amino group or at least one urethane group or at least two thereof as a refrigerant, for preparing a compound containing at least one ester group, for preparing a compound containing at least one ether group, for preparing a compound containing at least one amino group, for preparing a compound containing at least one urethane group, as a heat transfer medium, as a hydraulic fluid or as a component for a hydraulic fluid, for lowering the freezing point of aqueous phases, as a solvent or softener in coloring agents, in particular in printing inks, as a solvent or as an auxiliary in preferably liquid laundry detergents, as an additive in animal feeds, as a moisture retaining agent in foods and tobacco products, as a formulation component in cosmetic products, as an additive or base or both in pump means or lubricants or both or as a formulation component of anticorrosive agents.

The invention will now be described in greater detail with reference to non-limiting examples:

### EXAMPLES

### Example 1

### Preparation of a 1,2-Propanediol Phase

A 2 m-long reaction tube having an inner diameter of 25 mm and a 1 l volume was filled with copper chromite tablets (⅛″×⅛″) which were prepared in accordance with Example 1 of U.S. Pat. No. 4,982,020 and are also suitable for the hydrogenation of glycerides to form fatty alcohol and 1,2-propanediol. Firstly, activation was carried out with 1% hydrogen in nitrogen and subsequently hydrogenation was carried out at 200 bar, 225° C. and a glycerol throughput of 0.25 l/h in the reaction tube thus prepared.

The degree of reaction of the glycerol was determined by gas chromatography analysis of the hydrogenation effluent obtained during the hydrogenation. With regard to the components 1,2-propanediol, ethylene glycol, glycerol and water, the hydrogenation effluent had the following composition which was determined by integrating the signals of the gas chromatogram: 70% by weight of 1,2-propanediol, 0.6% by weight of ethylene glycol, 11.4% by weight of glycerol and 18% by weight of water.

### Example 2

### Preparation of a Mono Fatty Acid Ester

557.5 g (2 mol) of technical oleic acid (acid value 201.2) are mixed with 342.8 g of the 1,2-propanediol phase obtained in Example 1 (3.6 mol based on the alcohols contained in the 1,2-propanediol phase). After the addition of 0.25 g of tin oxalate, the mixture is heated under N₂ first to 175° C. and then the reaction temperature is increased to 200° C. within 3 hours. After a total of 4 hours, the reaction is terminated.

The mixture is then cooled to 135° C. and excess 1,2-propanediol removed by distillation under vacuum at max. 19 mbar in 30 minutes.

An oleic acid ester A is obtained. The product data of this ester can be inferred from Table 1.

### Comparative Example 1

### Preparation of a Mono Fatty Acid Ester

Example 2 is repeated, although 274 g (3.6 mol based on the 1,2-propanediol) of 99% 1,2-propanediol from BASF was used instead of the 1,2-propanediol phase from Example 1.

An oleic acid ester B is obtained. The product data of this ester can also be inferred from Table 1.

TABLE 1

| | 1,2- | | | | | | |
|---|---|---|---|---|---|---|---|
| Oleic acid ester | propanediol | AV¹⁾ | SV²⁾ | OHV³⁾ | IV⁴⁾ | Monoester⁵⁾ | Diester⁵⁾ |
| B | 99% | 0.46 | 175.6 | 124.4 | 83.1 | 69.5% | 28.4% |
| A | from | 0.8 | 167.5 | 173.0 | 81.1 | 67.0% | 26.8% |
| | Example 1 | | | | | | |

¹⁾Acid value
²⁾Saponification value
³⁾OH group value
⁴⁾Iodine value
⁵⁾According to gas chromatogram, percentage of surface area after derivatization with the silylating agent MSTFA

### Example 3

### Preparation of an Emulsion by Means of Oleic Acid Ester A as the Emulsifier

Oleic acid A obtained from Example 2 is used to prepare an emulsion of water and paraffin (emulsion 1) and an emulsion merely of water and oleic acid B (emulsion 2). The emulsions were prepared in a 25 ml measuring cylinder by means of a glass rod with ball, emulsification being carried out in each case for one minute.

In this case, the following amounts were used:

### Emulsion 1:

5 ml of oleic acid ester A
10 ml of water
5 ml of paraffin

### Emulsion 2:

5 ml of oleic acid ester A
10 ml of water

The properties of emulsions 1 and 2 can be inferred from Table 2.

### Comparative Example 2

### Preparation of an Emulsion by Means of Oleic Acid Ester B as the Emulsifier

Example 3 is repeated, oleic acid ester B being used instead of oleic acid ester A. Obtained in this case are emulsions 3 (water, oleic acid ester and paraffin) and 4 (water and oleic acid), the properties of which can also be inferred from Table 2.

TABLE 2

| Emulsion | 5 min | 15 min | 30 min | 18 h |
|---|---|---|---|---|
| 3 | ~6 ml | 8 ml | 10 ml | 10 ml |
| 1 | Strong | Strong | 0.5 ml phase | ~2 ml phase |
| | emulsion | emulsion | on top | on top |
| 4 | 10 ml | 10 ml | 10 ml | 10 ml |
| 2 | ~8 ml with | ~8 ml with | ~9 ml with | ~9 ml |
| | drops of fat | drops of fat | drops of flat | |

As may be seen from Table 2, those emulsions which were prepared using an oleic acid ester according to the invention are characterized by noticeably improved stability in storage.

### Example 4

### Preparation of a Further Emulsion by Means of Oleic Acid Ester A as the Emulsifier

Oleic acid A obtained from Example 2 is used to prepare an emulsion of water and soya oil (emulsion 5). For this purpose, ester and oil were mixed in the test tube using the emulsifying ball and subsequently water was incorporated in portions.

In this case, the following amounts were used:

### Emulsion 5:

3.0 g of oleic acid ester A
7.5 g of soya oil
10.0 g of water

### Comparative Example 3

### Preparation of a Further Emulsion by Means of Oleic Acid Ester B as the Emulsifier

Example 4 is repeated, oleic acid ester B being used instead of oleic acid ester A. In this case, emulsion 6 is obtained.

Whereas oleic acid ester B displays almost no emulsifier effect under the test conditions

(rapid separation into two phases took place), oleic acid ester A has a marked emulsifying effect (a creamy emulsion was obtained, displaying only slow rising of the organic phase).

### Examples 5-7 and Comparative Examples 4-6

### Preparation of a PVC Formulation by Means of Oleic acid Ester A or B as the Additive

The following PVC formulations were prepared:

TABLE 3

| Component/ | | | | Com. | Com. | Com. |
|---|---|---|---|---|---|---|
| parts | Ex. 5 | Ex. 6 | Ex. 7 | Ex. 4 | Ex. 5 | Ex. 6 |
| PVC Evipol | 100 | 100 | 100 | 100 | 100 | 100 |
| SH 5730¹⁾ | | | | | | |
| Mod. Kane | 5 | 5 | 5 | 5 | 5 | 5 |
| ACE B 58 A²⁾ | | | | | | |
| MARK 17 | 1.5 | 1.5 | 1.5 | 1.5 | 1.5 | 1.5 |
| MOK³⁾ | | | | | | |
| MOD. | 0.5 | 0.5 | 0.5 | 0.5 | 0.5 | 0.5 |
| PARALOID | | | | | | |
| K 120 N⁴⁾ | | | | | | |
| MOD. | 0.5 | 0.5 | 0.5 | 0.5 | 0.5 | 0.5 |
| PARALOID | | | | | | |
| K 175 ER⁵⁾ | | | | | | |
| Ca stearate | 0.1 | 0.1 | 0.1 | 0.1 | 0.1 | 0.1 |
| precipitated | | | | | | |
| Oleic acid | 1.0 | 5.0 | 10.0 | — | — | — |
| ester A | | | | | | |
| Oleic acid | — | — | — | 1.0 | 5.0 | 10.0 |
| ester B | | | | | | |

¹⁾Polyvinylchloride suspension from INEOS Vinyls Deutschland GmbH
²⁾Impact resistance modifier from Kaneca
³⁾Heat stabilizer from Crompton-Witco
⁴⁾Processing aid from Rohm and Haas Co
⁵⁾Processing aid from Rohm and Haas Co

The hardness (determined as Shore hardness A) was calculated from the PVC compositions thus obtained. In this case, it was ascertained that the compositions obtained in Examples 5 to 7 had the same hardness as the compositions obtained in Comparative Examples 4 to 6. Therefore, the esters according to the invention, which were obtained compared to the esters known in the art using a simplified process (no purification of the 1,2-propanediol phase formed during the partial hydrogenation of glycerol), are just as suitable as an additive in plastics material compositions as the esters of the prior art obtained using complex processes (use of previously purified 1,2-propanediol to prepare the esters).

1. A process for preparing a compound comprising a group selected from at least one ether group, at least one ester group, at least one amino group, at least one urethane group or at least two thereof, including the process steps:
a1) preparing 1,2-propanediol by means of a process wherein glycerol is hydrogenated in the presence of a catalyst, glycerol being reacted to at most 95% and a 1,2-propanediol phase is obtained; and
a2) reacting the 1,2-propanediol phase of step a1) with a compound comprising at least one active hydrogen atom, or at least one epoxide group, or at least one ester group, or at least one isocyanate group.

2. The process according to claim 1, wherein in process step a2) the 1,2-propanediol phase obtained in process step a1) is reacted with a compound comprising at least one carboxylic acid group so as to obtain a compound comprising at least one ester group.

3. The process according to claim 1, wherein a heterogeneous, copper and chromium containing catalyst is used in process step a1).

4. The process according to claim 3, wherein a heterogeneous copper chromite containing catalyst is used in process step a1).

5. The process according to claim 1, wherein the hydrogenation is carried out in process step a1) in tubular reactors or multitube fixed-bed reactors operated under isothermal conditions.

6. The process according to claim 1, wherein the hydrogenation is carried out in at least one reaction unit containing a reactor which is not operated under isothermal conditions and which is connected to a cooler.

7. The process according to claim 1, wherein the hydrogenation is carried out in process step a1) in such a way that liquid glycerol is passed in trickle bed operation in parallel, or countercurrent flow, with hydrogen over a catalyst fixed bed.

8. The process according to claim 5, wherein glycerol is passed in process step a1) through the catalyst charge in the reactor, or the reaction tubes, under measures at least partly preventing backmixing for a defined residence time.

9. The process according to claim 1, wherein the hydrogenation is carried out in process step a1) at a temperature in the range of from about 180 to about 280° C.

10. The process according to claim 1, wherein the water content of the glycerol used in process step a1) is below about 2%.

11. The process according to claim 1, wherein glycerol is reacted in process step a1) to at most 90%.

12. The process according to claim 1, wherein
the 1,2-propanediol phase obtained in process step a1) contains glycerol contamination in a range of from 0.01 to 20% by weight, based on the total amount of the 1,2-propanediol phase,
and
the 1,2-propanediol phase used in process step a2) contains at least 70% by weight of the glycerol contamination of the 1,2-propanediol phase.

13. The process according to claim 1, wherein the ratio of glycerol to 1,2-propanediol in the 1,2-propanediol phase obtained in process step a1) is in a range of from about 1:3 to about 1:8.

14. The process according to claim 1, wherein the 1,2-propanediol phase obtained in process step a1) is not purified prior to the reaction in process step a2).

15. The process according to claim 2, wherein the compound comprising at least one carboxylic acid group is a C₅ to C₃₀ monocarboxylic acid.

16. The process according to claim 15, wherein the compound comprising at least one carboxylic acid group which is a fatty acid.

17. The process according to claim 16, wherein the fatty acid is oleic acid.

18. A compound comprising a group selected from at least one ether group, at least one ester group, at least one amino group or at least one urethane group, wherein the group is produced by the process as set forth in claim 1.

19. The compound according to claim 18, wherein the compound is a fatty acid mono ester.

20. Use of chemical products, said use comprising a compound having a group selected from at least one ether group, at least one ester group, or at least one urethane group, preferably having at least one ester group, said compound produced according to claim 18.

21. Chemical products, containing comprising a compound containing a group selected from at least one ether group, at least one ester group or at least one urethane group, wherein the group is produced by the process as set forth in claim 1.

22. The chemical product according to claim 21 further comprising an emulsion wherein said emulsion comprises:
an aqueous phase;
an organic phase; and
a compound containing at least one ester group
wherein said aqueous phase is emulsified in said organic phase or said organic phase emulsified in said aqueous phase.

23. Process for preparing an emulsion, comprising the step of:
bringing an aqueous phase into contact with each other in the presence of a compound comprising at least one ester group produced according to the process comprising the steps of:
a1) preparing 1,2-propanediol by means of a process wherein glycerol is hydrogenated in the presence of a catalyst, glycerol being reacted to at most 95% and a 1,2-propanediol phase obtained; and
a2) reacting the 1,2-propanediol phase with a compound comprising at least one ester group.

24. An emulsion, produced according to the process of claim 23 further comprising the step of
bringing the aqueous phase and the organic phase into contact with each other in the presence of the compound containing at least one ester group.

25. A cosmetic composition, comprising the emulsion of claim 24.

26. A process for preparing a cosmetic composition, wherein the emulsion according to claim 23 is prepared and is brought into contact with at least one cosmetic component.

27. A plastics material composition comprising at least one plastics material and the compound of claim 18.

28. A process for preparing a plastics material composition, comprising the steps of:
bringing the compound of claim 18 into contact with at least one plastics material.

29. A drilling composition containing comprising at least one liquid flushing component and the compound of claim 18.

30. A process for preparing a drilling composition, comprising the step of:
bringing the compound of claim 18 into contact with at least one liquid flushing component.

31. Uses including a refrigerant, for preparing a compound containing at least one ester group, for preparing a compound containing at least one ether group, for preparing a compound containing at least one amino group, for preparing a compound containing at least one urethane group, as a heat transfer medium, as hydraulic fluid or as a component for a hydraulic fluid, for lowering the freezing point of aqueous phases, as a solvent or softener in colouring agents, in particular in printing inks, as a solvent or as an auxiliary in preferably liquid laundry detergents, as an additive in animal feeds, as a moisture retaining agent in foods and tobacco products, as a formulation component in cosmetic products, as an additive or base, or both, in pump media or lubricants or both or as a formulation component of anticorrosive agents, said uses comprising the 1,2-propanediol phase obtained in process step a1) of the process according to claim 1.

32. Use of the compound containing at least one ester group according to claim 16 as an emulsifier or as a formulation component for PVC extrusions.
